(19) United States
(12) Patent Application Publication
(10) Pub. No.: US 2023/0061931 A1
(43) Pub. Date: Mar. 2, 2023

(54) SPORTS GEAR VENTILATION BAG

(71) Applicant: Norm Otheim, West Fargo, ND (US)

(72) Inventor: Norm Otheim, West Fargo, ND (US)

(21) Appl. No.: 17/823,100

(22) Filed: Aug. 30, 2022

Related U.S. Application Data
(60) Provisional application No. 63/238,920, filed on Aug. 31, 2021.

(51) Int. Cl. F26B 9/00; A45C 3/00; A45C 15/00; A61L 9/01

(52) U.S. Cl. CPC F26B 9/003; A45C 3/00; A45C 15/00; A61L 9/01; A45C 2003/007

(57) ABSTRACT

A fan assembly for a sports bag includes an air distributor mountable to the faceplate and a fan mountable to the face plate to distribute air into the air distributor.

The present disclosure claims priority to U.S. Provisional Patent Disclosure Ser. No. 63/238,920, filed Aug. 31, 2021.

### BACKGROUND

This disclosure generally relates to sports equipment and more specifically to a bag that dries sports equipment and clothing.

Sports equipment and clothing often can become sweaty, smelly, wet, or otherwise soiled after use. Sports equipment and clothing is referred to as gear. Gear can also include other items of apparel or items of equipment that may become sweaty, smelly, wet, or otherwise soiled. When gear is put away in such conditions, mold and mildew contamination may occur. Commonly, the used gear is again placed in a bag for transportation after use.

### SUMMARY

This disclosure is directed toward a sports gear ventilation bag assembly for drying gear. The gear ventilation bag assembly may be used to begin the drying process, or it may be used to fully dry gear. The sports gear ventilation bag assembly is herein referred to as the “ventilation bag assembly.” The device is used to transport gear as well as dry the gear. Sports equipment and clothing are referred to herein as “gear.” Gear can also include other items of apparel or items of equipment that may become sweaty, smelly, wet, or otherwise soiled. Gear that is sweaty, smelly, wet, or otherwise soiled is herein referred to as “soiled.” Soiled gear which is not dried may develop mold and mildew conditions.

Gear that has become soiled due to use is placed in the ventilation bag assembly. The ventilation bag assembly is turned on such that a fan forces air to be circulated through the ventilation bag assembly. The air that is being circulated causes the gear to be dried. The circulation of air is the movement of air. The movement of air provides protection of equipment and clothing from mold and mildew. The mold and mildew may develop from sweat and water accumulation on the gear which is not dried. Air is circulated from the exterior of the ventilation bag assembly to the interior of the ventilation bag assembly.

Air that is circulated can also be infused with desired scents. For example, one might wish to infuse the circulated air with a clean scent when sports gear is within the ventilation bag assembly. Alternatively, one might wish to infuse the circulated air with a particular animal smell or scent concealment product when hunting gear is within the ventilation bag assembly.

A fan assembly for a bag according to one disclosed non-limiting embodiment of the present disclosure includes an air distributor mountable to the faceplate; and a fan mountable to the face plate to distribute air into the air distributor.

A further embodiment of any of the foregoing embodiments of the present disclosure includes that the face plate is mountable to a sports bag.

A further embodiment of any of the foregoing embodiments of the present disclosure includes that the face plate is mountable to a bag.

A further embodiment of any of the foregoing embodiments of the present disclosure includes that the air distributor is located at least partially within a bag.

A further embodiment of any of the foregoing embodiments of the present disclosure includes that the air distributor comprises a multiple of distribution ducts that extend within the bag, each of the multiple of distribution ducts comprising multiple exits.

A further embodiment of any of the foregoing embodiments of the present disclosure includes that the face plate is mountable to an interior of a bag and the fan is mountable to the face plate to sandwich a portion of the sports bag therebetween.

A further embodiment of any of the foregoing embodiments of the present disclosure includes a flange positioned against an exterior surface of the bag such that fasteners extend through the flange, through the exterior surface of the bag, and into the faceplate.

A further embodiment of any of the foregoing embodiments of the present disclosure includes a deodorizing system attached to the flange.

A further embodiment of any of the foregoing embodiments of the present disclosure includes that the deodorizing system comprises a space in the flange to receive a deodorant.

A further embodiment of any of the foregoing embodiments of the present disclosure includes that air is circulated from the fan through the slot, then to the air distributer.

A further embodiment of any of the foregoing embodiments of the present disclosure includes a deodorizing system attached to the faceplate.

A further embodiment of any of the foregoing embodiments of the present disclosure includes that the deodorizing system comprises a space in the faceplate to receive a deodorant.

A ventilation bag assembly according to one disclosed non-limiting embodiment of the present disclosure includes a bag; a faceplate mounted to the bag; an air distributor mountable to the faceplate; and a fan mountable to the face plate to distribute air into the air distributor.

A further embodiment of any of the foregoing embodiments of the present disclosure includes that the face plate is mountable to an interior of the bag and the fan is mountable to the face plate to sandwich a portion of the bag therebetween.

A further embodiment of any of the foregoing embodiments of the present disclosure includes that the air distributor is located at least partially within the bag.

A further embodiment of any of the foregoing embodiments of the present disclosure includes that the air distributor comprises a multiple of distribution ducts that extend within the bag.

A further embodiment of any of the foregoing embodiments of the present disclosure includes that the multiple of distribution ducts comprise four ducts.

A further embodiment of any of the foregoing embodiments of the present disclosure includes that the four ducts are arranged in a rectilinear pattern.

A further embodiment of any of the foregoing embodiments of the present disclosure includes that the four ducts are parallel to each other.

A further embodiment of any of the foregoing embodiments of the present disclosure includes a deodorizing system attached to the faceplate.

A further embodiment of any of the foregoing embodiments of the present disclosure includes that the deodorizing system comprises a space in the faceplate to receive a deodorant.

### BRIEF DESCRIPTION OF THE DRAWINGS

These and other features and advantages of the present invention will become more readily appreciated as the same becomes better understood by reference to the following detailed description when considered in connection with accompanying drawings, wherein:

FIG. 1 is a perspective of a ventilation bag assembly;

FIG. 2 is an exploded view of a ventilation bag assembly;

FIG. 3 is a side view of the ventilation bag assembly;

FIG. 4 is a ventilation bag assembly exploded view of a fan device for the bag.

### DETAILED DESCRIPTION

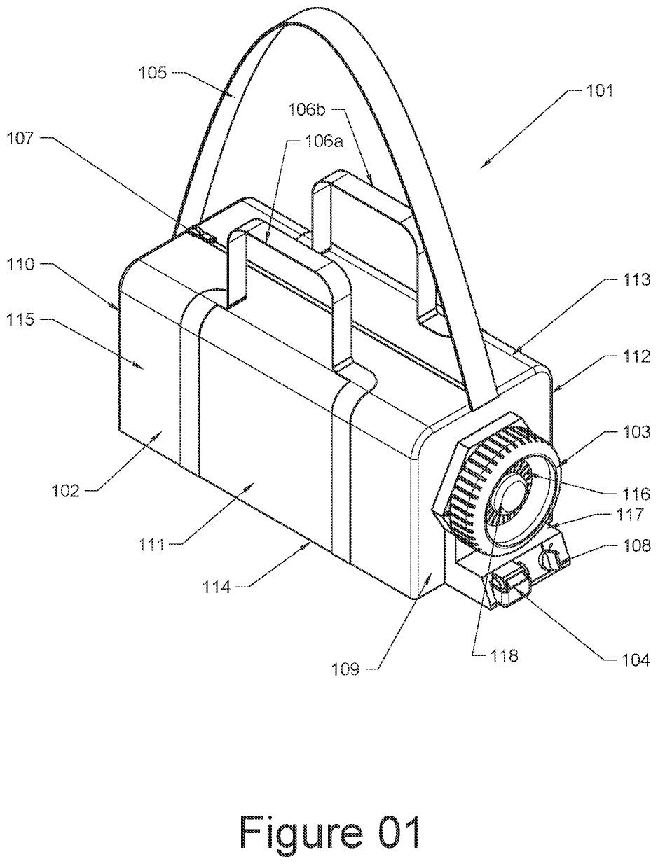

FIG. 1 is a perspective of a ventilation bag assembly 101. The ventilation bag assembly 101 comprises of a bag 102, a fan assembly 103, interior tubing, a battery 104, and an exit port.

The bag 102 in FIG. 1 is shown with a shoulder strap 105, hand straps 106 (individually labeled as 106a and 106b), a zipper 107, and a switch 108. The bag 102 is shown with a front end 109, a back end 110, a left side 111, a right side 112, a top 113, and a bottom 114. The bag 102 has an exterior surface 115 and an interior surface. Within the interior surface is the interior of the bag 102. Gear is placed within the interior through the zipper 107. The bag 102 may be various types of bags such as backpacks, running bags, yoga bags, etc. constructed of any type of material appreciated by a person of ordinary skill in the art. For example, the bag 102 may be constructed out of leather, cotton, nylon, cordura, denim, straw, synthetic rubbers, mesh, plastics, or other such material.

The fan assembly 103 includes a fan housing 117, a motor 118, the switch 108, fan blades, an intake 116, and an output. The fan assembly 103 is attached to the bag 102 on the front end 109. Alternatively, the fan assembly 103 may be attached to the back end 110, the left side 111, the right side 112, the top 113, or the bottom 114. The fan assembly 103 circulates air from the exterior of the bag 102 to the interior. The fan assembly 103 is configured such that the intake 116 of the fan assembly 103 is exposed to the air present at the exterior of the bag 102 and the output is connected to the interior tubing.

The battery 104 is electrically attached to the switch 108 which is electrically attached to a motor 118 which comprises the fan assembly 103. The battery 104 provides electric energy, which spins the motor 118. The motor 118 spins a fan blade which causes air to be circulated. The switch 108 may be configured to cause the motor 118 to spin at a variable speed or at preselected speeds. An example lithium-ion battery can be used as the battery 104. The battery 104 as shown in FIG. 1 may be disconnected from the fan assembly 103 using a quick-disconnect type connection. A quick-disconnect type connection is a connection in which the battery can be removed by activating one or more parts by hand. The battery 104 can be removed for the purpose of charging, reduction of weight, or replacement with an alternative battery. An alternating current power supply may be electrically connected to the fan assembly 103 to provide electrical energy.

The fan assembly 103 is sized to adequately circulate air through the bag 102. The interior tubing extends from the fan assembly 103 to the interior of the bag 102 such to ensure air reaches the extent of the bag 102. The exit port are openings on the bag 102 which allow for air to be circulated from the interior of the ventilation bag assembly 101 to the exterior of the ventilation bag assembly 101. In some embodiments, an exit port may be a slit or hole in the bag 102, which allows air to circulate. The slit or hole can be covered in a mesh material. Alternatively, the bag 102 may be constructed from a breathable material that allows air to circulate out of the bag 102 without the need for a slit or a hole. In the alternative embodiment, the exit port may essentially comprise of the entire bag material rather than a slit or a hole.

The ventilation bag assembly 101 may be configured in any size which is commonly used to contain gear. Examples of bag types which the ventilation bag assembly 101 may be equivalently configured include: hockey bags, boxing bags, ski bags, rugby bags, baseball bags, and other similar bags that contain gear.

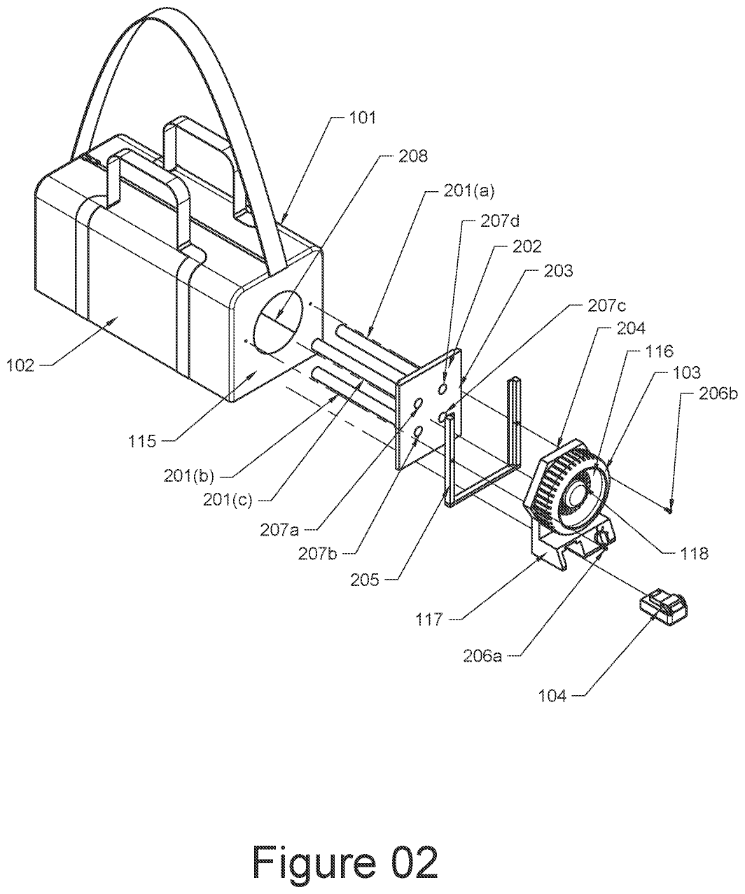

With reference to FIG. 2, the bag 102 is shown exploded from the fan assembly 103, the air distributer 202, and the battery 104. Additionally, a flange 205 and fasteners 206 (individually labeled 206a and 206b) are shown. An input hole 208 is cut from the bag 102. The hole as shown in FIG. 2 is circular, but it may be configured in any shape that allows for air to be circulated from the fan assembly 103 to the air distributer 202. The fan assembly 103 pulls air into the intake 116 and circulates it to the output 204. The motor 118 spins a fan blade which causes the air to flow.

An air distributer 202 includes one or more distribution ducts 201 (individually labeled as 201a, 201b, and 201c) and a faceplate 203 that supports the air distributer 202. The air distributer 202 receives air which is circulated from the output 204 of the fan assembly 103. The air distributer 202 delivers the circulated air to the extent of the interior of the bag 102. An air distributer 202 may be changed based on the desired application. An alternative embodiment of the air distributer 202 may comprise of a faceplate 203 with no distribution ducts attached. In this embodiment, air is simply circulated out of the faceplate holes 207 into the bag 102. The face plate is mountable to the interior of the sports bag and the fan is mountable to the face plate to sandwich a portion of the sports bag therebetween to retain the fan assembly 103 to the sports bag. A user need only cut a relatively small hole in their bag to install the fan assembly 103.

The distribution ducts 201 may be configured and shaped to deliver the air to any particular location within the interior of the bag 102. The distribution ducts 201 and the faceplate 203 may be connected to form an air distributer 202. The faceplate 203 is configured such that there are faceplate holes 207 (individually labeled as 207a, 207b, 207c, and 207d) where the distribution ducts 201 are connected. The faceplate holes 207 allow for air to be circulated through the faceplate holes 207 into the distribution ducts 201. The distribution ducts 201 are hollow such that they are shaped similar to a pipe and such that air may flow in one end of the distribution ducts 201 and out the other end.

The air distributer 202 is configured such that it is located in the interior of the bag 102. The faceplate 203 is positioned against the interior surface of the bag 102 such that air may circulate from the fan assembly 103, through the faceplate holes 207 into the distribution ducts 201. The flange 205 is positioned against the exterior surface 115 of the bag 102 such that fasteners 206 may extend through the flange 205, through the exterior surface 115 of the bag 102, and into the faceplate 203 which is located within the interior of the bag 102. When the fasteners 206 are installed, the flange 205, the bag 102, and the air distributer 202 is securely held together. The fasteners 206 may be of any type appreciated by a person of ordinary skill in the art. The fasteners 206 may also include latches, screws, rivets, bolts, or other such fasteners. The fan housing 117 attaches to the flange 205 or may be otherwise sewn into the bag.

In FIG. 2 the battery 104 is shown as removed from the fan housing 117. In normal operation, the battery 104 may be removed.

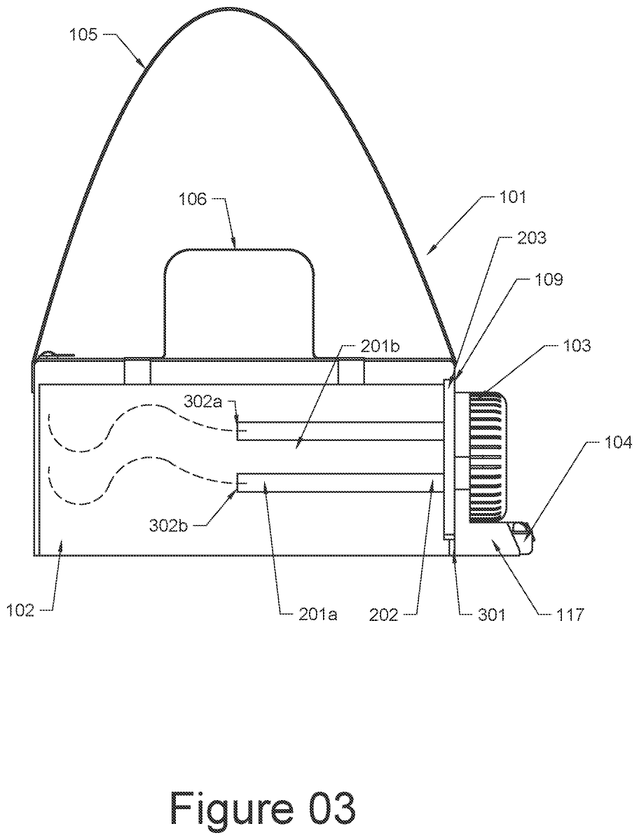

FIG. 3 is a perspective view of a ventilation bag assembly 101 showing a side profile. The fan assembly 103 is secured to the bag 102 on the front end 109. On the interior of the bag 102, the air distributer 202 is positioned against the interior surface 301 of the bag 102. More specifically, the faceplate 203 is positioned against the interior surface 301 of the bag 102. The faceplate 203 is secured to the flange 205 such that the fan assembly 103 and the air distributer 202 are secured together. The distribution ducts 201 extend from the faceplate 203 into the interior of the bag 102. The distribution ducts 201 may be configured such that their respective exhaust ends 302 (individually 302a and 302b) can be placed and directed at any location within the interior of the bag 102. Further, the distribution ducts 201 may be manufactured from a pliable material which may be manipulated by hand to a desired location within the bag 102 as gear is placed in the interior. For example, a distribution duct may be positioned to circulated air into a shoe.

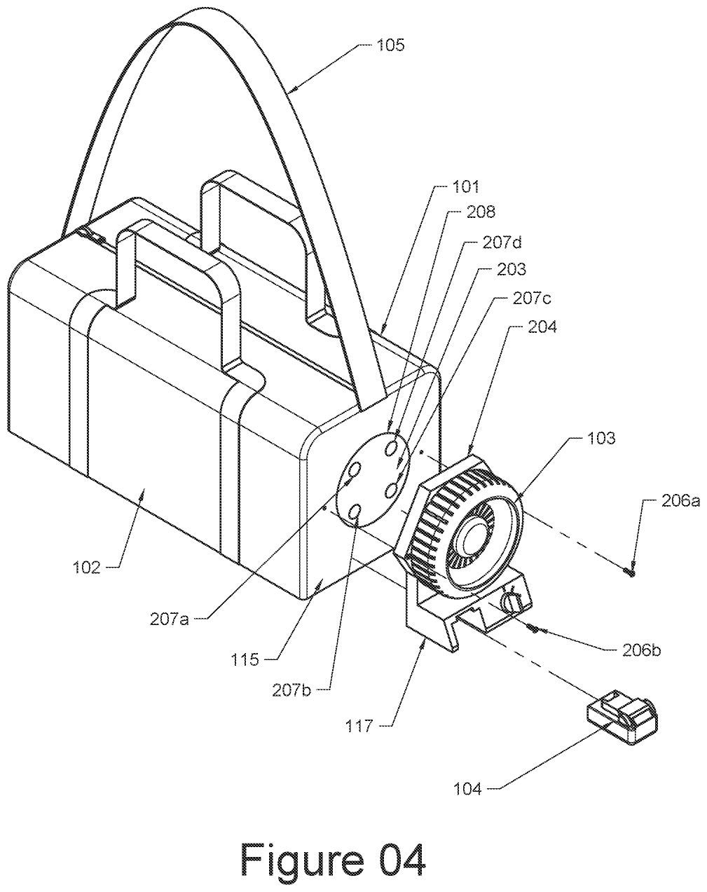

FIG. 4 is a perspective view of a ventilation bag assembly 101 showing the fan assembly 103 exploded from the bag 102. In the embodiment of FIG. 4 a flange 205 is not used. The fasteners 206 are extend through the fan housing 117, through the exterior surface 115, into the faceplate 203. In the figures, fasteners 206 are shown. Any number of fasteners 206 may be used which secure the fan assembly 103 to the faceplate 203. The output 204 of the fan assembly 103 is positioned such that air is circulated through the input hole 208, through the faceplate holes 207, and into the distribution ducts 201.

A deodorizing system may be attached to the ventilation bag assembly 101. The deodorizing system comprises a space wherein a deodorant solution or sheet could be placed within the bag 102. The space could be fitted on the flange 205, the bag 102, the fan housing 117, the faceplate 203, the air distributer 202, the intake 116, or any of the distribution ducts 201. One embodiment configures the space to be attached to the flange 205 such that air is circulated from the fan assembly 103, over or through the slot, and thence to the air distributer 202.

While various inventive aspects, concepts and features of the general inventive concepts are described and illustrated herein in the context of various exemplary embodiments, these various aspects, concepts, and features may be used in many alternative embodiments, either individually or in various combinations and sub-combinations thereof.

Unless expressly excluded herein all such combinations and sub-combinations are intended to be within the scope of the general inventive concepts. Still further, while various alternative embodiments as to the various aspects, concepts and features of the inventions (such as alternative materials, structures, configurations, methods, devices and components, alternatives as to form, fit and function, and so on) may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the general inventive concepts even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary, or representative values and ranges may be included to assist in understanding the present disclosure; however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated.

Although the different non-limiting embodiments have specific illustrated components, the embodiments of this invention are not limited to those particular combinations. It is possible to use some of the components or features from any of the non-limiting embodiments in combination with features or components from any of the other non-limiting embodiments.

The foregoing description is exemplary rather than defined by the limitations within. Various non-limiting embodiments are disclosed herein, however, one of ordinary skill in the art would recognize that various modifications and variations in light of the above teachings will fall within the scope of the appended claims. It is therefore to be appreciated that within the scope of the appended claims, the disclosure may be practiced other than as specifically described. For that reason, the appended claims should be studied to determine true scope and content.

What is claimed is:

1. A fan assembly for a bag, comprising
a faceplate;
an air distributor mountable to the faceplate; and
a fan mountable to the face plate to distribute air into the air distributor.

2. The fan assembly as recited in claim 1, wherein the face plate is mountable to a bag.

3. The fan assembly as recited in claim 1, wherein the air distributor is located at least partially within a bag.

4. The fan assembly as recited in claim 3, wherein the air distributor comprises a multiple of distribution ducts that extend within the bag, each of the multiple of distribution ducts comprising multiple exits.

5. The fan assembly as recited in claim 1, wherein the face plate is mountable to an interior of a bag and the fan is mountable to the face plate to sandwich a portion of the sports bag therebetween.

6. The fan assembly as recited in claim 5, further comprising a flange positioned against an exterior surface of the bag such that fasteners extend through the flange, through the exterior surface of the bag, and into the faceplate.

7. The fan assembly as recited in claim 6, further comprising a deodorizing system attached to the flange.

8. The fan assembly as recited in claim 7, wherein the deodorizing system comprises a space in the flange to receive a deodorant.

9. The fan assembly as recited in claim 8, wherein air is circulated from the fan through the slot, then to the air distributer.

10. The fan assembly as recited in claim 1, further comprising a deodorizing system attached to the faceplate.

11. The fan assembly as recited in claim 10, wherein the deodorizing system comprises a space in the faceplate to receive a deodorant.

12. A vent bag assembly, comprising
a bag;
a faceplate mounted to the bag;
an air distributor mountable to the faceplate; and
a fan mountable to the face plate to distribute air into the air distributor.

13. The ventilation bag assembly as recited in claim 12, wherein the face plate is mountable to an interior of the bag and the fan is mountable to the face plate to sandwich a portion of the bag therebetween.

14. The ventilation bag assembly as recited in claim 12, wherein the air distributor is located at least partially within the bag.

15. The ventilation bag assembly as recited in claim 14, wherein the air distributor comprises a multiple of distribution ducts that extend within the bag.

16. The ventilation bag assembly as recited in claim 15, wherein the multiple of distribution ducts comprise four ducts.

17. The ventilation bag assembly as recited in claim 16, wherein the four ducts are arranged in a rectilinear pattern.

18. The ventilation bag assembly as recited in claim 16, wherein the four ducts are parallel to each other.

19. The ventilation bag assembly as recited in claim 18, further comprising a deodorizing system attached to the faceplate.

20. The ventilation bag assembly as recited in claim 19, wherein the deodorizing system comprises a space in the faceplate to receive a deodorant.
